(19) United States
(12) Patent Application Publication
(10) Pub. No.: US 2026/0007789 A1
(43) Pub. Date: Jan. 8, 2026

(54) SANITATION SYSTEM FOR SANITIZING AN OBJECT BY MEANS OF SANITIZING ELECTROMAGNETIC RADIATION

(71) Applicant: ELEDRICITY, Lokeren (BE)

(72) Inventor: Kilian-Duncan VERSTRAETEN, Wetteren (BE)

(21) Appl. No.: 18/992,152

(22) PCT Filed: Jul. 7, 2023
(86) PCT No.: PCT/EP2023/068927

§ 371 Date: Jan. 7, 2025

(30) Foreign Application Priority Data
Jul. 7, 2022 (BE) BE2022/5556

(51) Int. Cl. A61L 2/10; A23B 2/53; A61L 2/24; A61L 2/26

(52) U.S. Cl. CPC A61L 2/10; A61L 2/24; A61L 2/26; A23B 2/53; A61L 2202/11; A61L 2202/122; A61L 2202/14; A61L 2202/24; A61L 2202/25

(57) ABSTRACT

A sanitation system for sanitizing an object is disclosed. The system comprises a support surface having a target location arranged to receive the object; an illumination module arranged to radiate the target location with sanitizing electromagnetic radiation (“SER”); a dosimeter module comprising at least a first dosimeter arranged to measure a dose of SER emitted by the illumination module and received by the dosimeter (the “measured dose”); a database storing instructions indicating the required dose of SER to reach the target location (the “required dose”); and a control module in communication with the illumination module, the database and the dosimeter module. The control module is arranged to receive from the dosimeter module the level of the measured dose. The control module is further arranged to control, based on the level of the measured dose, the illumination module such that the required dose reaches the target location.

### CROSS-REFERENCE TO RELATED APPLICATION(S)

This present application is a national stage application of International Patent Application No. PCT/EP2023/068927, filed Jul. 7, 2023, which claims priority to Belgium Patent Application No. 2022/5556, filed Jul. 7, 2022, the disclosures of which are hereby incorporated by reference in their entireties.

### TECHNICAL FIELD

The present disclosure relates to a sanitation system for sanitizing an object by means of sanitizing electromagnetic radiation. The present disclosure further relates to a method for sanitizing an object by means of sanitizing electromagnetic radiation, the method comprising using the above mentioned sanitation system.

### Background

Sanitation of objects such as medical instruments, foodstuff or writing utensils has become an integral part of our daily activities. A common method of sanitizing the objects is simple cleaning in soap and water. This proves however to be insufficient to kill sufficient pathogens on the object. Traditional cleaning with water also may be ineffective for items which cannot be completely submerged, such as wood. Other approaches which involve the use of chemicals and extreme heat to sanitize the objects are somewhat more effective than traditional cleaning with water, but both practices have their disadvantages. For example, heat may damage the object or may require a cool-down period before the object can be used, while the use of chemicals can often lead to chemical residues left on the objects. Furthermore, the emerging resistance to biocides makes this a more imminent problem.

It is known that pathogens can be inactivated or destroyed by irradiation with electromagnetic radiation of specific wavelengths. Such radiation is also referred to as sanitizing electromagnetic radiation (“SER”), also known as non-ionizing radiation or non-ionizing germicidal radiation. For example, ultraviolet light, in particular of the UV-C range, can efficiently inactivated or destroy many different types of pathogens. The required dose to inactivate or destroy the pathogen depends on the pathogen in question. Many different studies have been performed to determine the required doses. A particular dose can be delivered to the object, and thus to the pathogens on the object, by irradiating the object with SER of a particular radiation irradiance for a particular radiation duration. The irradiance of the SER reaching the object depends on the intensity of the SER emitting illumination module and the distance of the object to said illumination module.

It has however been found by the present inventors that typical current SER sanitation systems, in order to guarantee that the required dose is delivered to the object, produce a very high amount of SER radiation such as to deliver an overdose to the pathogens. These systems work without taking into account the systems energy efficiency, illumination module lifetime or illumination module defects, which according to the present disclosure could be used to guarantee the delivery of the required dose to the pathogens on the object whilst limiting the amount of overdose being delivered.

### SUMMARY

The present disclosure aims to provide a SER based sanitation system wherein the delivery of the required dose to the pathogen on the object is guaranteed whilst limiting the amount of overdose being delivered.

To that end, the present disclosure provides a sanitation system for sanitizing an object according to the first claim. The sanitation system comprises the following elements:
a support surface having a target location arranged to receive the object to be sanitized. In some embodiments the object to be sanitized is placed on the exposed surface of the support surface, i.e. on the surface of the support surface which is facing the illumination module as introduced below. In some embodiments the object to be sanitized is the exposed surface of the support surface itself. This will be made clear in the use cases described in the brief description of the figures;
an illumination module arranged to radiate the target location with sanitizing electromagnetic radiation (further referred to as “SER”);
a dosimeter module comprising at least a first dosimeter, for example a first dosimeter and a second dosimeter as exemplified further below in the present description. The first dosimeter is arranged to measure a dose of SER. The SER is emitted by the illumination module and received by the first dosimeter. This dose that is measured by the first dosimeter is further referred to as the “measured dose”;
a database storing instructions indicating the required dose of SER to reach the target location (further referred to as the “required dose”). The required dose typically depends on the type of pathogen which needs to be inactivated or destroyed. It is for example possible for a user to indicate to the system, for example through a user interface panel, the type of pathogen that needs to be targeted, for example virus, bacteria, fungi or spores, wherein the first two typically require lower doses than the latter two. Alternatively, the system could for example comprise detection means arranged to detect the type of pathogen that needs to be targeted; and
a control module in communication with the illumination module, the database and the dosimeter module, the control module being arranged to receive from the first dosimeter the level of the measured dose, and further being arranged to control, based on the level of the measured dose, the illumination module such that the required dose reaches the target location.

This sanitation system allows to control the illumination module based on insight on the dose that is effectively being delivered to the object, i.e. as opposed to blindly overdosing the object as is done in the prior art. This sanitation system in particular enables to provide a feedback loop between the dosimeter module and the illumination module.

According to a first example, the system for example enables to measure in real time what dose is being delivered to the object i.e. by the intermediary of the dose being measured by the first dosimeter, and to turn off the illumination module only when the required dose is delivered to the object. Given that the first dosimeter and the object are positioned at a predetermined position with respect to the illumination module, and that the dose depends on the radiation duration, the radiation intensity, and the distance between the illumination module and the first dosimeter or object, it is possible to link the measured dose to a dose received by the object. Indeed, from the measured dose, the radiation duration and the distance between the illumination module and the first dosimeter one can calculate the intensity of radiation emitted by the illumination module. From the radiation intensity, based on the distance between the illumination module and the object and the radiation duration, one can calculate the dose received by the object. In other words, the control of the illumination module in the present example comprises turning off the illumination module when the measured dose reaches a predetermined critical level indicating that the dose delivered to the target location has reached the required dose.

According to a second example, additionally or alternatively to the above mentioned first example, the sanitation system for example enables to measure whether the illumination module radiates the expected dose towards the object, i.e. the dose that would be expected upon driving the illumination module at a given power setting for a given radiation duration. For example, upon detecting that the expected dose is not reached in the given radiation duration, one can conclude that for some reason the power delivered to the illumination module is not transformed in the expected radiation intensity. This could for example be a consequence of an occlusion of the illumination module for example due to dirt having accumulated on the illumination module. The control module can take this information into account when controlling the illumination module, i.e. by increasing the power setting of the illumination module such as to increase the radiation intensity and/or by increasing the radiation duration. The sanitation system is able to determine the non-receiving of the expected dose at the object, by means of the first dosimeter. This is because, similarly to the object, the first dosimeter also expects to receive a particular dose upon being radiated by an illumination module powered at a particular power setting for a given radiation duration. If this expected dose does not correspond to the actual measured dose by the first dosimeter, than one can conclude that for some reason the power delivered to the illumination module is not transformed in the expected radiation intensity. In other words, the control of the illumination module comprises comparing the level of measured dose (i.e. measured by the first dosimeter) with an expected dose to reach the first dosimeter upon running the illumination module for a predetermined amount of time at a predetermined power setting, and further comprises adapting, if this would be deemed required based on the comparison of the measured dose and the expected dose, one of the power setting of the illumination module and/or the duration that the illumination module emits the SER. The above mentioned procedure could for example be performed as a calibration step prior to radiating the object. The calibration step could for example comprise turning on the illumination module for a predetermined calibration time, for example ten seconds, at a predetermined calibration power setting, for example at one watt, and measuring with the first dosimeter the dose that is received by the first dosimeter. Given that the first dosimeter is positioned at a predetermined position with respect to the illumination module, and that the measured dose depends on the radiation intensity, the distance between the illumination module and the first dosimeter, as well as the radiation duration, one can calculate what dose is expected to be measured by the first dosimeter based on a predetermined relation between the power setting of the illumination module and the radiation intensity emitted by the illumination module. If however, the expected dose is not measured, then the predetermined relation between the power setting and the radiation intensity is no longer correct and needs to be adapted. The adapted relation enables the control module to determine the power setting that is needed to obtain the radiation intensity that is required to radiate the object with the required dose in a required radiation duration. Alternatively, the control module could maintain the power setting of the illumination module and use the adapted relation to determine the adapted radiation intensity and to control the radiation duration such that the required dose is delivered to the object. Instead of performing a one-time calibration of the sanitation system prior to sanitizing the object as described above, the illumination module and the dosimeter module can also be operated as a repeating feedback loop. The first dosimeter can for example determine the measured dose multiple times during the radiation of the object, and adapt the relation between the power setting of the illumination module and the radiation intensity accordingly during use. This enables for example to take into account instantaneous occluding of the illumination module. The first dosimeter can for example determine a measured dose at least once every two seconds, preferably at least once every second, more preferably at least ten times every second. The predetermined radiation duration is then for example equal to the period of sampling or equal to a fraction, for example half, of the period of sampling. The predetermined power setting would then not be a “calibration power setting” as described above, but would be the power setting as applied during radiation of the object which power setting is prior to the taking of the sample considered to be the correct power setting based on the earlier relation between power setting of the illumination module and the radiation intensity. The second example as described above is now further exemplified with three use cases.
1) The power setting of the illumination module could be adjusted during the annual onsite service. The service technician for example notices in the logbook that the time to reach the required dose is increasing. This means that the illumination module is producing less radiation, for example due to occlusion of the illumination module or for example due to defects of the illumination module such as ageing defects. The service technician then for example increases the power setting of the illumination module in order to reduce the time required for reaching a certain dose.
2) The control module could automatically change the power setting of the illumination module according to a benchmark dose which is measured while starting up the device. The control module for example includes a startup routine where the illumination module is asked to provide a radiation dose ‘x’ to the first dosimeter in ‘y’ seconds. If this dose was not given in ‘y’ seconds, the startup routine is repeated with a ‘z’ percent higher power setting of the illumination module until the desired dose ‘x’ is given in the required time ‘y’.
3) If the system has an increased intelligence it could be able to predict the end time of radiation for example by measuring 10 times a second. If this predicted/extrapolated end-time is more than the maximum time set, the control module could increase the power setting of the illumination module in order to reduce the radiation duration such as to reach the required dose in the predetermined radiation duration.

According to an embodiment of the present disclosure, the SER is UVC radiation, preferably having a peak wavelength between 180 and 280 nanometers, preferably between 240 and 280 nanometers, preferably between 260 and 275 nanometers.

According to an embodiment of the present disclosure, the illumination module is a LED-based illumination module. It has been found by the present inventors that a LED based illumination module enables to easily change power setting of the illumination module and thus the radiation intensity emitted by the illumination module. LEDs can after all easily be dimmed, for example by dimming the LED driver by varying the input signal between 0-10V. This is pretty unusual in the existing UVC world. Indeed, in the existing UVC world most of the illumination modules used are discharge lamps which are very difficult to dim (if possible, at all). As described above, the dimming of the illumination module can be controlled by the control module in order to guarantee the delivery of the required dose to the object. The dimming functionality could for example be used to overcome the aging effect of the LEDs. Preferably, a new illumination module will be shipped with the led driver dimmed at 80%, thus allowing to increase the power setting of the illumination module when required.

According to an embodiment of the present disclosure, the system further comprises the object positioned on the target location. The object is for example a medical instrument such as a stethoscope.

According to an embodiment of the present disclosure, the system is arranged for disinfecting the object.

According to an embodiment of the present disclosure, the first dosimeter, and preferably the entire dosimeter module, i.e. the first dosimeter, but also any further dosimeter that are part of the dosimeter module, is positioned at the side of the support surface opposite to the side where the illumination module is provided, and wherein the support surface is made of material that is transparent to the SER.

According to an embodiment of the present disclosure, the system further comprises a box enclosing the support surface, the illumination module and the dosimeter module. The box is preferably arranged to prevent SER from leaving the box. Preferably, the interiors of the walls of the box are made of a material that is reflective for the SER. This allows to increase the SER irradiation on the object. Preferably, the system comprises a drawer removable from the box, wherein the support surface is provided in the drawer. This enables to easily place and remove objects on the target location on the support surface.

According to an embodiment of the present disclosure, the dosimeter module comprises a further dosimeter in addition to the above mentioned first dosimeter. The further dosimeter is referred to as the second dosimeter. In further embodiments a further dosimeter could still be provided in the dosimeter module, wherein the embodiments described below with respect to the first and second dosimeter, apply mutatis mutandis to the first, second and further dosimeters. The second dosimeter is provided at a physically distinct location from the first dosimeter. The second dosimeter is for example provided on the opposite side of the object with respect to the first dosimeter. Preferably, the distance between the first dosimeter and the second dosimeter, preferably of all the dosimeters in the dosimeter module, is at least 5 cm, preferably at least 10 cm, preferably at least 15 cm. Preferably, the first and second dosimeter, preferably all the dosimeters in the dosimeter module, lie in a plane referred to as the “dosimeter plane”. Preferably the dosimeter plane lies parallel to the plane of the support surface. Preferably, the first and second dosimeters, preferably all the dosimeters of the dosimeter module, lie on the opposite side of the supporting surface with respect to the illumination module. Similar to the first dosimeter, the second dosimeter is arranged to measure a dose of SER emitted by the illumination module and received by the second dosimeter (further referred to as the “second measured dose”). The control module is in communication with the second dosimeter, and is arranged to receive from the second dosimeter the level of the second measured dose. The control module is arranged to control, based on the level of the first and second measured dose, the illumination module such that the required dose reaches the target location. Providing two dosimeters has the advantage of providing more information to the control module regarding the state of sanitation of the object, thereby enabling to more accurately determine when the object has received the required dose. It has for example been found by the present inventors that objects, in particular irregularly shaped objects, tend to reflect SER differently in different directions. It has in particular been found that it is possible that a dosimeter receives SER directly from the illumination module as well as from a reflection on the object. That dosimeter thus detects a measured dose which is higher than what would be expected to be received based only on the direct irradiation by the illumination module. It is thus desired to obtain a measured dose at the dosimeter which is substantially only the result of direct irradiation from the illumination module. The present embodiment enables to obtain a better detection of such a measured dose substantially related to direct irradiance. By providing two dosimeters, one could for example take an average of the first and second measured doses in case the distance between the illumination module and the first and second dosimeters are equal. If these distances are not equal, one has to take into account the respective distances to the illumination module, for example by calculating for each measured dose an estimate of the radiation intensity emitted by the illumination module and by averaging both estimated radiation intensities. The averaged radiation intensity could then for example be used to determine the dose received by the object based on the distance between the illumination module and the object and based on the radiation duration. It is for example possible to use a ‘dosimeter conversion factor’ for each dosimeter to compensate for the different readings in measured doses due to the difference in distance to the illumination module. With these conversion factors, one can directly compare the measured doses of both dosimeters although they are at different distances to the illumination module. In a particular embodiment of the present disclosure, only the reading of the dosimeter that has the lowest irradiation by reflected SER is used. One could for example use the above mentioned ‘dosimeter conversion factor’, and only use the dosimeter with the lowest measured dose as corrected by the dosimeter conversion factor. This could for example also be implemented by calculating an estimate of the radiation intensity emitted by the illumination module based on the first and second measured doses, i.e. by determining a first estimate radiation intensity based on the first measured dose and a second estimate radiation intensity based on the second measured dose, and by only taking the reading of the dosimeter that estimates the lowest radiation intensity. Equivalently, this could for example also be implemented by determining the dose received by the object based on the first and second measured doses, i.e. the first dosimeter predicts that the object has received dose X and the second dosimeter predicts that the object has received dose Y, and by only taking the reading of the dosimeter that predicts the lowest received dose, i.e. the first dosimeter if dose X is lower than dose Y. In other words, the control module is arranged to select from the levels of the first and second measured dose, the measured dose that indicates the lowest level of dose being delivered to the target location (further referred to as the “selected measured dose”), and wherein the control module is further being arranged to control, based on the level of the selected measured dose, the illumination module such that the required dose reaches the target location.

According to an embodiment of the present disclosure, the illumination module comprises an array of light elements, also referred to as photonic radiator elements. The light elements are for example LEDs, for example specifically arranged to emit SER for example UVC radiation. Preferably, the light elements in the array are connected in series. Preferably, the array is a planar array, i.e. forming a plane. The plane for example lies parallel to the support surface. wherein the planar array determines a “plane of the illumination module”. In case multiple dosimeters are provided in the dosimeter module, for example the above mentioned first and second dosimeters, and in case said multiple dosimeters lie in a plane, then preferably the “plane of the illumination module” lies parallel to the plane of the dosimeters. This embodiment has the advantage that the distance between the illumination module and the dosimeters are equal, which enables to directly compare the measured doses of the dosimeters as described above.

According to an embodiment of the present disclosure, the system further comprises a defect monitoring module in communication with the illumination module and the control module. The defect monitoring module is arranged to detect a defect in one of the light elements in the array and to communicate a detected defect to the control module. The defect is for example one of a short-circuit of the light element, an ageing problem of the light element or a temperature problem of the light element. The present embodiment enables the control module to control the illumination module based on the communicated detected defect. The control module for example controls the illumination module by adjusting one of the power setting of the illumination module or the duration of the SER radiation. This control is for example a control of the entire illumination module or for example only of the defective light elements. An example of control by the control module comprises adapting the aforementioned relation between the power setting of the illumination module or of the defective part of the illumination module and the radiation intensity emitted by the illumination module or by the defective part of the illumination module. Another example of a control by the control module comprises deactivating the entire illumination module or the defective part of the illumination module upon detecting a defect. Simultaneously the control module could emit a warning signal to the user of the sanitation system to prompt the user to service the sanitation system. Furthermore, the present embodiment has the particular advantage that one can provide a limited number of dosimeters, for example the above described two dosimeters, to monitor a section of the illumination module, for example only parts of the array of light elements, and to extrapolate the finding of the dosimeters to the entire illumination module in case no defects are detected.

According to an embodiment of the present disclosure, the defect monitoring module is arranged to measure the voltage over the array of light elements and to compare the measured voltage with an expected voltage. The expected voltage can be determined based on the arrangement of light elements in the array and the power settings of the illumination module. The array of light elements is preferably a series connection of the light elements as described above. In case the light elements are LEDs as described above, then the voltage over the series arrangement of LEDs is a multiple of the forward voltage of each LED. Preferably, in particular when the illumination module comprises an array of series connected LEDs, the defect monitoring module communicates a defect to the control module when the comparison of the measured voltage with the expected voltage differs by at least one time the forward voltage of the LED. Preferably, the defect monitoring module communicates a short-circuit defect to the control module when the measured voltage is lower than the expected voltage by at least one time the forward voltage of the LED. Preferably, the defect monitoring module communicates an ageing or temperature defect to the control module when the measured voltage is higher than the expected voltage by at least one time the forward voltage of the LED. The control module could use the knowledge of the type of defect to control the illumination module. The control module could also inform the user of the sanitation system of the type of defect and recommend a corresponding type of servicing. The system described in patent publication U.S. Pat. No. 10,939,528, in particular as shown in FIGS. 9 and 10, is an example of voltage based defect monitoring system. This system is therefore incorporated herein by reference.

According to an embodiment of the present disclosure, the support surface is transparent to SER, and a further, i.e. second, illumination module is provided on the opposite side of the support surface with respect to the above mentioned illumination module, further referred to as the first illumination module. The embodiments described above with respect to the first illumination module preferably apply mutatis mutandis to the second illumination module. For example, the second illumination module preferably also comprises a planar array of light elements. Preferably, the plane of the first illumination module lies parallel to the plane of the second illumination module. Preferably, also a further, i.e. second dosimeter module comprising at least one dosimeter, is provided in addition to the above mentioned dosimeter module, further referred to as the first dosimeter module. The second dosimeter module is preferably provided on the opposite side of the support surface with respect to the first dosimeter module. The embodiments described above with respect to the first dosimeter module preferably apply mutatis mutandis to the second dosimeter module. For example, the second dosimeter module preferably also comprises multiple dosimeters lying in a plane. Preferably, the plane of the dosimeters in the first dosimeter module lies parallel to the plane of the dosimeters of the second dosimeter module. According to an embodiment of the present disclosure, the second dosimeter module relates to the second illumination module, in the same manner as how the first dosimeter module relates to the first illumination module, i.e. the embodiments described above with respect to the relation between the first illumination module and the first dosimeter module apply mutatis mutandis to the relation between the second illumination module and the second dosimeter module.

It is a further object of the present disclosure to provide for a method for sanitizing an object. The method comprises the step of providing the sanitation system as described above. The method comprises using the sanitation system. Preferably, the method further comprises providing an object that is to be sanitized, and placing the object within the target location on the support surface. The method preferably further comprises radiating the object with SER by means of the illumination module. The method preferably further comprises measuring the measured dose by means of the first dosimeter. The method preferably further comprises communicating the measured dose to the control module. The method preferably further comprises controlling by means of the control module, based on the level of the measured dose, the illumination module such that the required dose reaches the target location.

### BRIEF DESCRIPTION OF THE FIGURES

FIG. 1 is a schematic view of a sanitation system according to an embodiment of the present disclosure.

FIG. 2 is a schematic view of a sanitation system according to a further embodiment of the present disclosure wherein two dosimeters are provided.

FIGS. 3a and 3b show a schematic view of a sanitation system according to a further embodiment of the present disclosure wherein the system further comprises a defect monitoring module.

FIG. 4 is a schematic view of the sanitation system according to a first use case.

FIG. 5 is a schematic view of a variant of the sanitation system 1 shown schematically in FIG. 2, and illustrates a second use case.

FIG. 6 is a schematic view of a variant of the sanitation system 1 shown schematically in FIG. 1, and illustrates a third use case.

FIG. 7 is a schematic view of a variant of the sanitation system 1 shown schematically in FIG. 1, and illustrates a fourth use case.

FIG. 8 is a schematic view of a variant of the sanitation system 1 shown schematically in FIG. 2, and illustrates a fifth use case.

### DETAILED DESCRIPTION

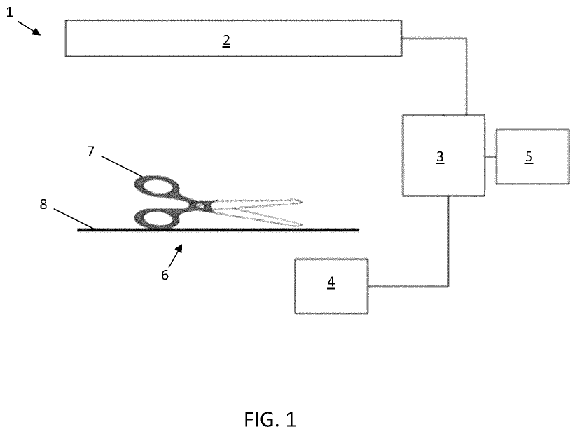

FIG. 1 is a schematic view of a sanitation system 1 according to an embodiment of the present disclosure. The sanitation system 1 comprises the following elements:
a support surface 8 having a target location 6 arranged to receive an object 7 to be sanitized. In the present example the object is a scissor;
an illumination module 2 arranged to radiate the target location with sanitizing electromagnetic radiation (further referred to as “SER”); The illumination module 2 comprises an array of light elements. The light elements are preferably LEDs specifically arranged to emit SER as UVC radiation.

The light elements in the array are connected in series and form a planar array, i.e. forming a plane. The plane lies parallel to the support surface 8;
a dosimeter module comprising a first dosimeter 4 arranged to measure a dose of SER. The SER is emitted by the illumination module and received by the first dosimeter. This dose that is measured by the first dosimeter is further referred to as the “measured dose”;
a database 5 storing instructions indicating the required dose of SER to reach the target location (further referred to as the “required dose”). The required dose typically depends on the type of pathogen which needs to be inactivated or destroyed; and
a control module 3 in communication with the database 5, the illumination module 2 and the dosimeter module, the control module being arranged to receive from the first dosimeter 4 the level of the measured dose, and further being arranged to control, based on the level of the measured dose, the illumination module such that the required dose reaches the target location.

This sanitation system allows to control the illumination module based on insight on the dose that is effectively being delivered to the object, i.e. as opposed to blindly overdosing the object as is done in the prior art. This sanitation system in particular enables to provide a feedback loop between the dosimeter module and the illumination module.

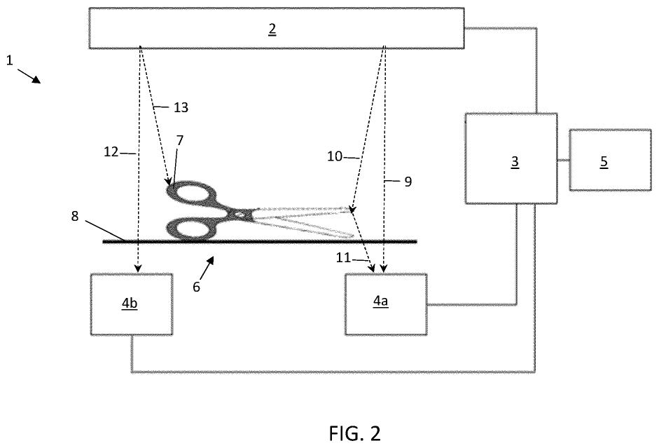

FIG. 2 is a schematic view of a sanitation system 1 according to a further embodiment of the present disclosure wherein two dosimeters are provided in the dosimeter module. The elements that are in common with the sanitation system shown in FIG. 1, are given the same reference number. The sanitation system 1 shown in FIG. 2 differs from the sanitation system shown in FIG. 1 in that the dosimeter module comprises a further dosimeter 4b in addition to the above mentioned first dosimeter 4a. The further dosimeter is referred to as the second dosimeter 4b. The second dosimeter 4b is provided at a physically distinct location from the first dosimeter, in particular on the opposite side of the object 7 with respect to the first dosimeter 4a. The first and second dosimeter 4a, 4b lie in a plane referred to as the “dosimeter plane”. This dosimeter plane lies parallel to the plane of the support surface 8. The first and second dosimeters 4a, 4b lie on the opposite side of the supporting surface 8 with respect to the illumination module 2 and the support surface is therefore made substantially transparent to SER. Similar to the first dosimeter 4a, the second dosimeter 4b is arranged to measure a dose of SER emitted by the illumination module 2 and received by the second dosimeter (further referred to as the “second measured dose”). The control module is in communication with the second dosimeter, and is arranged to receive from the second dosimeter the level of the second measured dose. The control module is arranged to control, based on the level of the first and second measured dose, the illumination module such that the required dose reaches the target location. Providing two dosimeters has the advantage of providing more information to the control module regarding the state of sanitation of the object, thereby enabling to more accurately determine when the object has received the required dose. It has for example been found by the present inventors that objects, in particular irregularly shaped objects such as the illustrated scissor, tend to reflect SER differently in different directions. This is illustrated by two incoming SER rays 13 and 10, of which ray 13 is absorbed by the object and ray 10 is reflected by the object in reflected ray 11. It has in particular been found that it is possible that a dosimeter such as dosimeter 4a in the figure receives SER directly from the illumination module through illustrated ray 9 as well as from a reflection on the object through illustrated ray 11. That dosimeter 4a thus detects a measured dose which is higher than what would be expected to be received based only on the direct irradiation by the illumination module 2. It is thus desired to obtain a measured dose at the dosimeter which is substantially only the result of direct irradiation from the illumination module. The present embodiment enables to obtain a better detection of such a measured dose substantially related to direct irradiance. By providing two dosimeters 4a, 4b, one could for example take an average of the first and second measured dose, or one could only use the reading of the dosimeter that has the lowest measured dose.

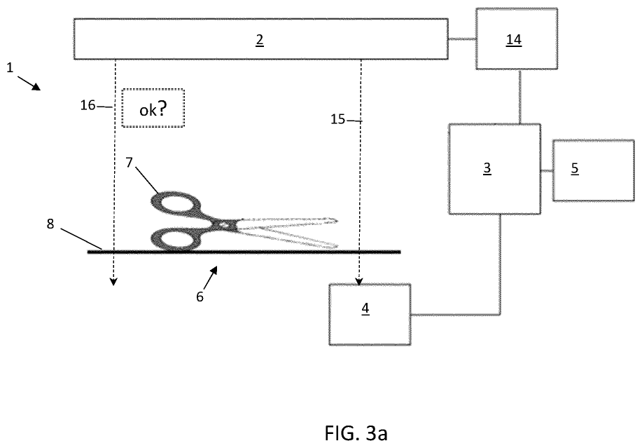

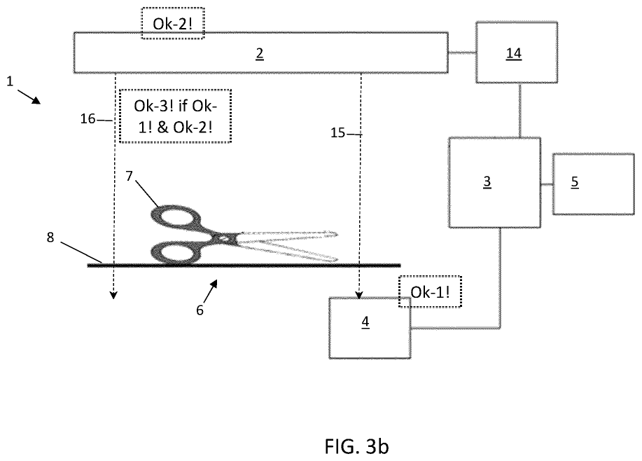

FIGS. 3a and 3b show a schematic view of a sanitation system 1 according to a further embodiment of the present disclosure. The elements that are in common with the sanitation system shown in FIG. 1, are given the same reference number. The sanitation system 1 shown in FIG. 3 differs from the sanitation system shown in FIG. 1 in that wherein the system further comprises a defect monitoring module 14. The defect monitoring module 14 is in communication with the illumination module 2 and the control module 3. The defect monitoring module 14 is arranged to detect a defect in one of the light elements in the array and to communicate a detected defect to the control module. The defect is for example one of a short-circuit of the light element, an ageing problem of the light element or a temperature problem of the light element. The present embodiment enables the control module to control the illumination module based on the communicated detected defect. The control module 3 controls the illumination module by adjusting one of the power setting of the illumination module or the duration of the SER radiation. The present embodiment has the particular advantage that one can provide a limited number of dosimeters, for example only one dosimeter as shown in FIG. 1 or the two dosimeters 4a, 4b as shown in FIG. 2, to monitor a section of the illumination module 2, i.e. only parts of the array of light elements, and to extrapolate the finding of the dosimeters to the entire illumination module in case no defects are detected. This process is shown in FIGS. 3a and 3b, wherein only the right half of the illumination module 2 is being monitored by first dosimeter 4, i.e. the left half of the illumination module is not directly monitored by a dosimeter. The first dosimeter gives information to the control module 3 regarding the delivery of the required dose by the monitored section of the illumination module 2, that is by the right half section of the illumination module. If one wants to know whether the findings of the first dosimeter 4 also apply to the other sections of the illumination module 2, that is the left half section of the illumination module (indicated in FIG. 3a by the boxed question “OK?”), one can rely on the observation of the first dosimeter 4 (indicated in FIG. 3b by the boxed statement “OK—1!”) and of the indication that all light elements of the illumination module 2 function correctly (indicated in FIG. 3b by the boxed statement “OK—2!”) to extrapolate the observation of the first dosimeter to the not-directly observed section of the illumination module, that is the left half section of the illumination module 2 (indicated in FIG. 3b by the boxed statement “Ok—3! if Ok—1! & Ok—2!”).

In the next figures, five different applications of the sanitation system of the present disclosure are presented. The different applications are referred to as use cases.

### Use Case 1—Disinfection Box

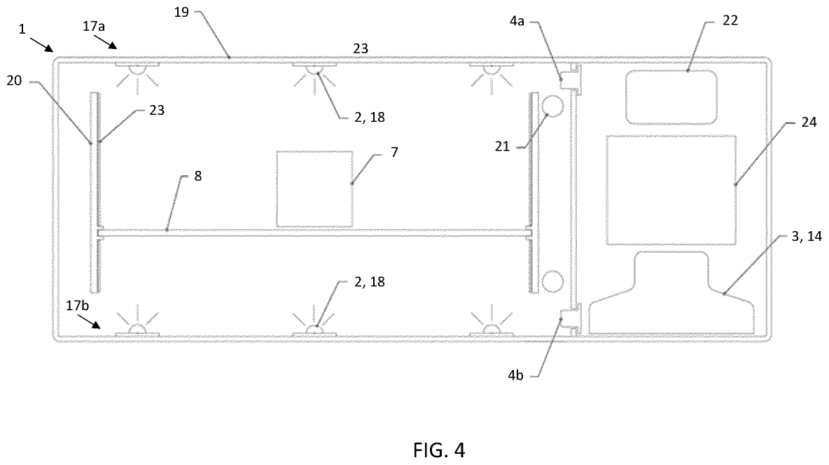

FIG. 4 is a schematic view of a variant of the sanitation system 1 shown schematically in FIG. 2 and illustrates a first use case. The elements that are in common with the sanitation system shown in FIG. 2, are given the same reference number. The sanitation system 1 shown in FIG. 4 differs from the sanitation system shown in FIG. 2 in that the illumination module 2 comprises two planar arrays 17a, 17b, the two planar arrays facing each other and being provided on opposite sides of the support surface 8. The support surface 8 is made out of Quartz glass which is substantially transparent to SER. The planar arrays 17a, 17b are formed by interconnection of six strips 18 of light elements. Each strip extends into the plane of the drawing. The illumination module 2 is reducing the amount of generated heat towards the object by being mounted on cooling surfaces. These surfaces remove the thermal energy from the illumination module by means of thermal conduction. The system 1 also comprises a box 19 enclosing the support surface 8, the illumination module 2 and the dosimeters 4a, 4b. The box functions as the above mentioned cooling surfaces. The box is arranged to prevent SER from leaving the box. The interiors of the walls of the box are made of a material that is reflective for the SER as indicated by reference number 23. This allows to increase the SER irradiation on the object. The system 1 further comprises a drawer 20 removable from the box, wherein the support surface 8 is provided in the drawer. This enables to easily place and remove objects on the target location on the support surface. The interior walls of the drawer are considered part of the interior walls of the box, and are thus provided with the above mentioned reflective surface. The drawer is moveable in and out of the plane of the drawing. The system 1 further comprises two door sensors 21 arranged to detect the open or closed configuration of the door of the box. The door sensors 21 prevent the operation of the system I until the system can be safely used, i.e. until the door of the box is closed by inserting the drawer into the box. To that end, the door sensors are in communication with the control module 3. Furthermore, the FIG. 4 shows the dimmable LED driver 22 in communication with the control module 3. The dimmable LED driver 22 is configured for providing current/voltage to the LEDs. The other use cases described below also comprise a dimmable (LED) driver, although this is not shown in the corresponding figures. Finally, the FIG. 4 also shows a user interface 24 implemented as a touchscreen, in communication with the control module 3. The user interface allows the user to start or stop the decontamination process. The present use case in particular shows a disinfection box 19 enclosing an object 7 that is radiated by UVC radiation during a flexible amount of time, the required time depending on the intensity of the UVC illumination module 2. The object is for example a stethoscope, or a hand-scanner for scanning items in a warehouse. The object is placed on the exposed surface of the support surface 8. The database 5 is not explicitly shown, and is considered to be integrated with the control module 3. The control module 3 uses two UVC dosimeters 4a, 4b to measure 10 times per second the radiation received from the illumination module 2. The “required UVC dose”, that is the UVC dose required to eliminate pathogens, is read into the control module 3 from the database 5. Both dosimeters 4a, 4b measure a dose at every sampling period, and accumulate these doses to obtain a value called “the measured dose”. Once the dosimeter 4a, 4b with the lowest measured dose has received a measured dose that surpasses a threshold indicating that the required dose is delivered to the object, the control module switches off the illumination module 2. If the time to achieve a given UVC dose ‘x’ becomes longer due to the illumination module 2 becoming older and radiating less, due to the illumination module becoming dirty by means of dust, or due to the supply voltage of the mains being low, for example when used in rural areas with non-optimal electricity grids, then the control module 3 can decide to increase the pre-dimmed power setting of the illumination module 2 in order to reduce the time to achieve dose ‘x’. The service engineer can also decide to change the illumination module 2 power setting by means of changing a value in a configuration file which causes the control module 3 to alter the power setting of the illumination module 2. For example, the service engineer can access the log file stored in the database, from which the service engineer can see the runtime of the UVC illumination module 2 as well as the configuration file with all preset values. The control module 3 can also be self-learning: if the pre-set power setting of the illumination module 2 is for example 80% and the required dose is for example 100mJ/cm2 and the pre-set desired time of radiation to achieve this dose is 3 minutes, the control module can decide to increase the power setting if 10% of the required radiation dose is not given in 10% of the pre-set time. The control module 3 for example increases the power setting of the illumination module 2 in order to arrive at 20% of the required dose being in 20% of the pre-set desired time. The control module 3 provides power to the illumination module 2 through an external safety relay. This safety relay can interrupt the power supply of the UVC illumination module 2 if errors occur. One of the errors is the above mentioned door sensor indicating that the door of the box 19 is opened. Another error is a radiation timing which exceeds the maximum radiation setting. Another error is an abnormal high UVC radiation intensity or abnormal fast radiation dose cycle. Another error is an abnormal voltage measured on the UVC light elements of the illumination module 2. The latter functions as follows. Multiple light elements, in the present example multiple LEDs, are connected in series such as to form the illumination module 2. The nominal voltage over the light elements is set in the database connected to the control module 3. This nominal voltage is the result of multiplying the individual required light element voltage with the amount of light elements connected. A defect monitoring module 14 is provided between the control module 3 and the illumination module 2 (the defect monitoring module 14 is shown as being incorporated in the control module 3). The defect monitoring module 14 measures the voltage over the light elements 10 times per second. When the measured voltage differs more than the voltage required by one light element, the defect monitoring module 14 informs the control module 3 of the measurement, upon which the control module 3 produces an error message indicating a defective light element.

### Use Case 2—Medical Room Disinfection

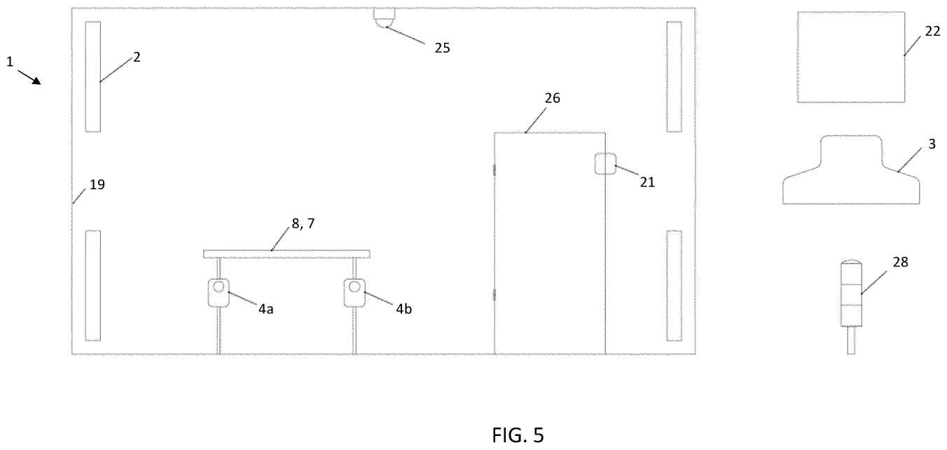

FIG. 5 is a schematic view of a variant of the sanitation system 1 shown schematically in FIG. 2, and illustrates a second use case. The elements that are in common with the sanitation system shown in FIG. 2, are given the same reference number. The present use case relates to a medical room such as an ICU (intensive care unit) room or an OR (operating room). The medical room is equipped with a UVC illumination module 2 comprising UVC discharge lamps. The walls of the medical room form a box 19 which is part of the sanitation system 1 of the present disclosure. The walls of the box 19 prevent SER from leaving the sanitation system. The illumination module 2 is shown as a fixed installation. Alternatively, the illumination module 2 might be a mobile UVC source for example provided on a robot that is moving around. In the medical room, some positions have been defined where disinfection needs to reach a predefined level, i.e. where the required dose needs to be delivered. One of those positions is the operation table, which forms a support surface 8 according to the present disclosure. In this case, the exposed surface of the operation table is the object to be sanitized. The delivery of the desired UVC disinfection level can be measured by using the sanitation system 1 of the present disclosure provided with the dosimeters. Two UVC dosimeters 4a, 4b are provided in the room close to the supporting surface. The dosimeters 4a, 4b are connected by cables or wireless, to the control module 3. The control module 3 itself is shown as being external to the room, however it could equally be positioned inside of the room. The database 5 is not explicitly shown, and is considered to be integrated with the control module 3. The dosimeters 4a, 4b will measure a UVC dose and will provide a signal to the control module 3 when the required dose has been achieved at the support surface 8 as described above in the first use case. In the same time the control module 3 monitors the safety of operation by using input of a presence detector 25 which sends out a stop signal to the control module 3 if a person is inside of the room, upon which the control module 3 controls the illumination module 2 such as to stop irradiation. Additionally a door sensor 21 (similar to the door sensor from the first use case) is added to switch off the radiation of the illumination module 2, by intermediary of the control module 3, if the door 26 giving access to the medical room is opened. The system 1 is activated by a remote touchscreen 22 placed next to the door 26 at the outside of the medical room. Furthermore, next to the door 26 there is a color-changing light strip 28. This light-strip indicates if it is safe to enter the room. If the system 1 is radiating SER, it is considered unsafe to enter the room and the light strip will indicate this, for example by means of a red light. The sanitation system 1 of the present use case is operated in a similar manner as the sanitation system of the first use case described above. The defect monitoring module 14 as described in the first use case is preferably also provided in the present use case.

### Use Case 3—Water or Air Sanitation Device

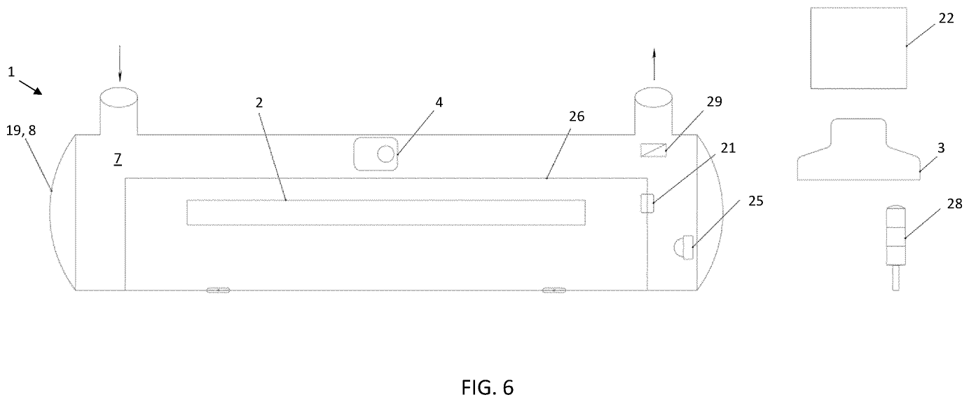

FIG. 6 is a schematic view of a variant of the sanitation system 1 shown schematically in FIG. 1, and illustrates a third use case. The elements that are in common with the sanitation system shown in FIG. 1, are given the same reference number. The present use case relates to a water or air reactor for sanitizing water or air by means of the sanitation system of the present disclosure. The reactor walls 19 are part of the sanitation system and form ducts that enable passage of the water or air. In the present use case, these ducts are the support surface 8. The object to be sanitized in the present use case is water or air. The reactor walls form a box which comprises an inlet opening and an outlet opening for allowing water or air to flow through the reactor. These openings are respectively indicated by means of an incoming arrow and an outgoing arrow. The walls of the box prevent SER from escaping the box. The reactor is provided with the illumination module 2 which might comprise LEDs or mercury discharge lamps. The control module 3 itself is shown as being external to the reactor, however it could equally be positioned inside of the reactor. The database 5 is not explicitly shown, and is considered to be integrated with the control module 3. The UVC dosimeter 4 is connected by a cable or wireless, to the control module 3 such that the control module 3 can control the operation of the illumination module 2 based on the measured dose of the dosimeter 4 as described above. For example the control module 3 increases the power setting of the illumination module 2 when the measured dose by the dosimeter 4 indicates that the required dose is not given to the object for the given flow rate of the water or air. This flow rate can for example be measured by means of a flow sensor 29 in communication with the control module 3. At the same time the control module 3 monitors the safety of operation by providing a presence sensor 25 within the reactor, which presence detector sends out a stop signal to the control module 3 if a human being is detected within the reactor, upon which the control module 3 shuts down the radiation operation of the illumination module 2. Additionally a door sensor 21 is added to switch off the radiation of the illumination module 2 by intermediary of the control module 3, if the reactor door 26 is opened. The system is activated by a remote touchscreen 22, positioned next to the reactor. Also next to the reactor there is a color-changing light strip 28. This light-strip indicates if the system is radiating, in which case it would be unsafe to open the reactor door 26. In case the control module 3 turns off the illumination module 2 due to the presence of an unsafe situation, the control module 3 also switches off the circulation pump in order to save energy by not having a pump run idle. The defect monitoring module 14 as described in use case 1 is preferably also provided in the present use case.

### Use Case 4—Food Industry Disinfection

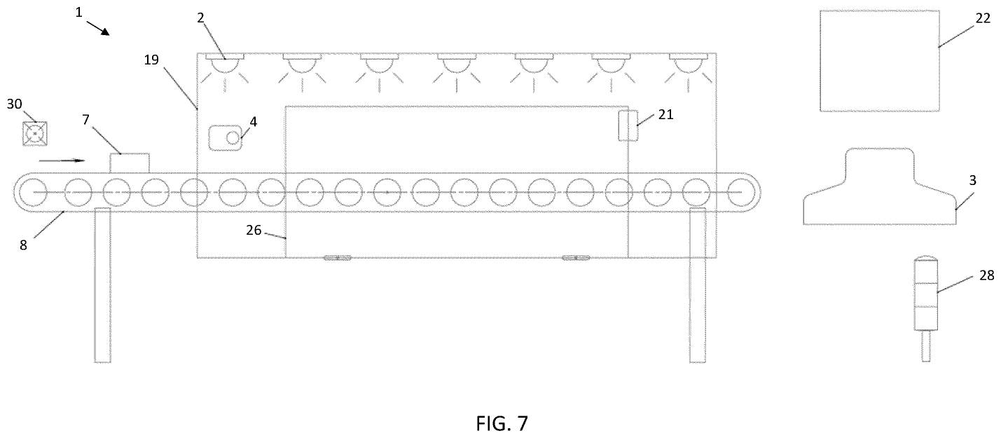

FIG. 7 is a schematic view of a variant of the sanitation system 1 shown schematically in FIG. 1, and illustrates a fourth use case. The elements that are in common with the sanitation system shown in FIG. 1, are given the same reference number. The present use case relates to a food disinfection conveyor belt for transporting foodstuff and for simultaneously sanitizing the foodstuff by means of the sanitation system 1 of the present disclosure. The food disinfection conveyor belt comprises a housing 19 arranged to prevent SER from leaving the housing. The conveyor belt runs through the housing with the exposed surface moving in a direction as indicated by the arrow. Entry and exit hatches (not shown) are provided in the housing to allow passage of the conveyor belt. The housing is part of the sanitation system 1 of the present disclosure, and the conveyor belt is the support surface 8. The object 7 to be sanitized in the present use case are foodstuff which are placed on the exposed surface of the support surface. The control module 3 itself is shown as being external to the housing, however it could equally be positioned inside of the housing. The database 5 is not explicitly shown, and is considered to be integrated with the control module 3. The housing is provided with the illumination module 2 which might comprise LEDs or mercury discharge lamps. The UVC dosimeter 4 is connected by a cable or wireless, to the control module 3 such that the control module 3 can control the operation of the illumination module 2 based on the measured dose of the dosimeter 4 as described above. For example the control module 3 increases the power setting of the illumination module 2 when the measured dose by the dosimeter 4 indicates that the required dose is not given to the object for the given conveyor belt speed. This conveyor belt speed can for example be measured by means of a conveyor belt movement detector 30 in communication with the control module 3. At the same time the control module 3 monitors the safety of operation by providing a presence detector within the housing, which presence detector sends out a stop signal to the control module 3 if a human being is detected within the housing, upon which the control module 3 shuts down the radiation operation of the illumination module 2. Additionally a door sensor 21 is added to switch off the radiation of the illumination module 2 by intermediary of the control module 3, if the housing door 26 is opened. The system is activated by a remote touchscreen, positioned next to the reactor. Also next to the reactor there is a color-changing light strip. This light-strip indicates if the system is radiating, in which case it would be unsafe to open the housing door. In case the control module 3 turns off the illumination module 2 due to the presence of an unsafe situation, the control module 3 also switches off the conveyor belt. The defect monitoring module 14 as described in use case 1 is preferably also provided in the present use case.

### Use Case 5—Payment Terminal Disinfection

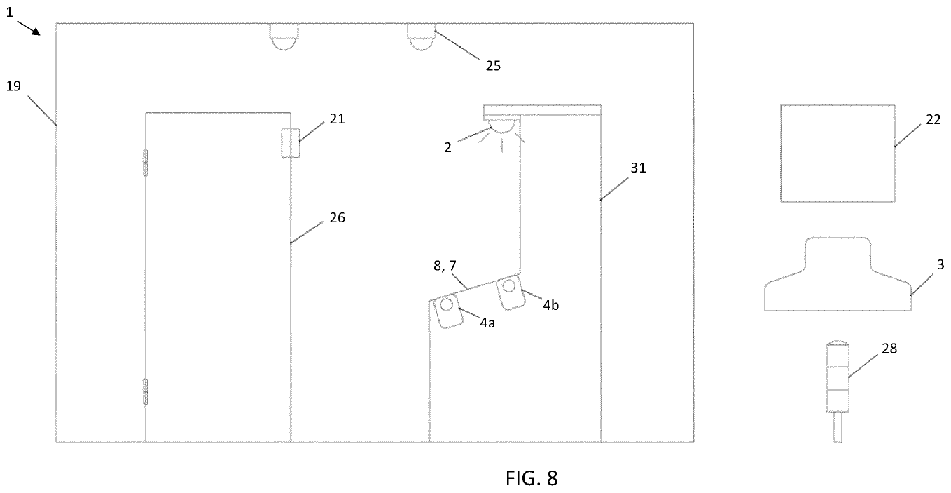

FIG. 8 is a schematic view of a variant of the sanitation system 1 shown schematically in FIG. 2, and illustrates a fifth use case. The elements that are in common with the sanitation system shown in FIG. 2, are given the same reference number. The present use case relates to a payment device 31 such as an ATM (automatic teller machine) or a payment terminal equipped with a UVC illumination module 2 comprising UVC discharge lamps or UVC LEDs. This illumination module 2 is directed towards the payment device keyboard where disinfection needs to reach a predefined level, i.e. where the required dose needs to be delivered. The keyboard forms the support surface 8 according to the present disclosure. The object to be sanitized in the present use case is the exposed surface of the support surface 8. The delivery of the desired UVC disinfection level can be measured by using the sanitation system 1 of the present disclosure provided with the dosimeters. Two UVC dosimeters 4a, 4b are provided in the payment device next to the keyboard. The dosimeters 4a, 4b are connected by cables or wireless, to the control module 3. These dosimeters 4a, 4b will measure the UVC dose indicative of the UVC dose delivered to the keyboard and will provide a signal to the control module 3 when the required dose has been achieved, similar to the operation of the dosimeter feedback as described in the first use case. The payment device is positioned inside of a room, for example an ATM room in a bank. The walls of the room form the housing 19 of the sanitation system 1. The control module 3 itself is shown as being external to the housing 19, however it could equally be positioned inside of the housing. The database 5 is not explicitly shown, and is considered to be integrated with the control module 3. The control module 3 also monitors the safety of operation of the sanitation system 1 by using input of a presence detector 25 which send out a stop signal to the control module if a person is entering the room, upon which the control module controls the illumination module 2 such as to stop irradiation. Additionally a door sensor 21 is added to switch off the radiation of the illumination module 2, by intermediary of the control module 3, if the door 26 giving access to the room is opened. The system 1 is activated by closing the door of the room and by a presence detector detecting that there is no human presence in the room. Furthermore, next to the door 26 on the outside of the room there is a color-changing light strip 28. This light-strip 28 indicates if it is safe to enter the room. If the system is detecting radiation it is considered unsafe to enter the room. The sanitation system 1 of the present use case is operated in a similar manner as the sanitation system of the first use case described above. The defect monitoring module 14 as described in use case 1 is preferably also provided in the present use case.

1. A sanitation system for sanitizing an object, the system comprising:
a support surface having a target location arranged to receive the object to be sanitized;
an illumination module arranged to radiate the target location with sanitizing electromagnetic radiation (further referred to as “SER”);
a dosimeter module comprising at least a first dosimeter arranged to measure a dose of SER emitted by the illumination module and received by the dosimeter (further referred to as the “measured dose”);
a database storing instructions indicating a required dose of SER to reach the target location (further referred to as the “required dose”); and
a control module in communication with the illumination module, the database and the dosimeter module, the control module being arranged to receive from the dosimeter module a level of the measured dose, and further being arranged to control, based on the level of the measured dose, the illumination module such that the required dose reaches the target location,
wherein the control of the illumination module comprises comparing the level of measured dose with an expected dose to reach the first dosimeter upon running the illumination module for a predetermined amount of time at a predetermined power setting, and further comprises adapting, based on a comparison of the measured dose and the expected dose, one of the power setting of the illumination module and/or a duration that the illumination module emits the SER, and
wherein the illumination module is a LED-based illumination module.

2. The system according to the preceding claim 1, wherein the illumination module comprises an array of light elements, wherein the light elements in the array are connected in series.

3. (canceled)

4. (canceled)

5. (canceled)

6. The system according to claim 2, wherein the system further comprises as defect monitoring module in communication with the illumination module and the control module, the defect monitoring module being arranged to detect a defect in one of the light elements in the array and to communicate a detected defect to the control module.

7. The system according to claim 6, wherein the control module is arranged to control the illumination module based on the communicated detected defect.

8. The system according to claim 7, wherein the control comprises adjusting one of the power setting of the illumination module or the duration of the SER radiation, preferably of the defective light elements.

9. The system according to claim 7, wherein the control comprises deactivating the illumination module.

10. The system according to claim 6, wherein the defect is one of a short-circuit of the light element, an ageing problem of the light element or a temperature problem of the light element.

11. The system according to claim 6, wherein the defect monitoring module is arranged to measure the voltage over the array of light elements and to compare the measured voltage with an expected voltage.

12. The system according to claim 2, wherein the light elements are LEDs, and wherein the defect monitoring module communicates a defect to the control module when the comparison of the measured voltage with the expected voltage differs by at least one time the forward voltage of the LED.

13. The system according to claim 12,
wherein the system further comprises as defect monitoring module in communication with the illumination module and the control module, the defect monitoring module being arranged to detect a defect in one of the light elements in the array and to communicate a detected defect to the control module,
wherein the defect monitoring module communicates a short-circuit defect to the control module when the measured voltage is lower than the expected voltage by at least one time the forward voltage of the LED, and
wherein the defect monitoring module communicates an ageing or temperature defect to the control module when the measured voltage is higher than the expected voltage by at least one time the forward voltage of the LED.

14. The system according to claim 1, wherein the control of the illumination module comprises turning off the illumination module when the measured dose reaches a predetermined critical level indicating that the dose delivered to the target location has reached the required dose.

15. (canceled)

16. The system according to claim 1, wherein the control module is arranged to perform the comparison of the measured dose and the expected dose and to perform the adjustment of the power setting of the illumination module or the duration of UVC radiation, during the sanitation of the object in a repeating feedback loop arrangement.

17. The system according to claim 16, wherein the comparison and adjustment steps are performed at least once every two seconds, preferably at least once every second.

18. (canceled)

19. (canceled)

20. (cancelled)

21. (canceled)

22. (canceled)

23. (canceled)

24. (canceled)

25. The system according to claim 1,
wherein the dosimeter module comprises a further, i.e. second, dosimeter,
wherein the second dosimeter is provided at a physically distinct location from the first dosimeter,
wherein the second dosimeter is arranged to measure a dose of SER emitted by the illumination module and received by the second dosimeter (further referred to as the “second measured dose”),
wherein the control module further is in communication with the second dosimeter,
wherein the control module is arranged to receive from the second dosimeters the level of the second measured dose, and
wherein the control module is arranged to control, based on the level of the first and second measured dose, the illumination module such that the required dose reaches the target location.

26. The system according to claim 25, wherein the control module is arranged to select from the levels of the first and second measured dose, the measured dose that indicates the lowest level of dose being delivered to the target location (further referred to as the “selected measured dose”), and wherein the control module is further being arranged to control, based on the level of the selected measured dose, the illumination module such that the required dose reaches the target location.

27. (canceled)

28. (canceled)

29. (canceled)

30. (canceled)
